(19) United States
(12) Patent Application Publication
(10) Pub. No.: US 2013/0137921 A1
(43) Pub. Date: May 30, 2013

(54) PROPELLING SYSTEM AND CAPSULE APPLYING THE SAME

(71) Applicants: Ludovic Angot, Hsinchu City (TW); Chun-Te Wu, Taoyuan County (TW); Yi-Jen Fang, Changhua County (TW)

(72) Inventors: Ludovic Angot, Hsinchu City (TW); Chun-Te Wu, Taoyuan County (TW); Yi-Jen Fang, Changhua County (TW)

(73) Assignee: INDUSTRIAL TECHNOLOGY RESEARCH INSTITUTE, Hsinchu (TW)

(21) Appl. No.: 13/561,093

(22) Filed: Jul. 30, 2012

Related U.S. Application Data
(60) Provisional application No. 61/563,842, filed on Nov. 28, 2011.

(51) Int. Cl. A61B 1/045

(52) U.S. Cl. 600/109

(57) ABSTRACT

A propelling system disposed in a chamber of a capsule is provided. The propelling system includes a mass and a damping module. The mass is configured for vibrating in the chamber along a plurality of directions. The damping module is coupled between the mass and the capsule for absorbing the kinetic energy of the mass, the damping module provides the smallest damping effect along a first direction. The disclosure further provides a capsule, which includes a shell having a chamber and at least one aforementioned propelling system disposed in the chamber. Accordingly, the capsule can be autonomously propelled by the propelling force autonomously produced by the propelling system.

### CROSS-REFERENCE TO RELATED APPLICATION

This application claims the priority benefit of U.S. provisional application Ser. No. 61/563,842, filed on Nov. 28, 2011. The entirety of the above-mentioned patent application is hereby incorporated by reference herein and made a part of this specification.

### TECHNICAL FIELD

The disclosure generally relates to a propelling system and a capsule employing the propelling system.

### BACKGROUND

In recent years, thanks to advances in medical technology, a number of instruments capable of examining the inside of the human body and assist to the detection of diseases have been developed. These inspecting and detecting instruments such as the endoscope are medical equipments which penetrates the body through various channels to observe the body's internal state. A typical endoscope includes a thin and elongated optical lens which can enter the human body from an existing channel of the body (for example, the oesophagus) or from a channel in the body established through surgery. By inserting the endoscope into the body, not only can images of the internal body be available to a surgeon, but also tissue can be repaired and malignant tumours removed. Flexible endoscopes are available to inspect the digestive system, but either the patient must be totally anaesthetized or some discomfort is felt. Also, flexible endoscopes don't allow the inspection of important parts of the digestion system, such as the small colon. Capsule endoscopes are miniature observation systems which are swallowed by the patient and they allow the complete observation of the whole digestive system.

However, some current capsule endoscopes are semi-autonomous, and they rely on an external device to provide magnetic fields as a power source to drive the capsule endoscope or to control its movement.

### SUMMARY

An exemplary embodiment of the disclosure is directed to a propelling system, which is disposed in a chamber of a capsule and includes a mass and a damping module. The mass is configured for vibrating in the chamber along a plurality of directions. The damping module is coupled between the mass and the capsule for absorbing the kinetic energy of the mass, the damping module provides the smallest damping effect along one preferential direction among the other directions of vibration.

An exemplary embodiment of the disclosure is also directed to a capsule, which includes a shell and at least one propelling system. The shell has a chamber, and the propelling system is the above-mentioned propelling system disposed in the chamber.

Several exemplary embodiments accompanied with figures are described in detail below to further describe the disclosure in detail.

### BRIEF DESCRIPTION OF THE DRAWINGS

The accompanying drawings constituting a part of this specification are incorporated herein to provide a further understanding of the disclosure. Here, the drawings illustrate embodiments of the disclosure and, together with the description, serve to explain the principles of the disclosure.

FIG. 1 is a side view diagram of a capsule according to the first exemplary embodiment of the disclosure.

FIG. 2 is the top view diagram of the capsule of FIG. 1.

FIG. 3A is a top view diagram of the capsule of FIG. 1 at the propelling system thereof.

FIG. 3B is another top view diagram of the capsule of FIG. 1 at the propelling system thereof.

FIG. 4A is a side view diagram of the capsule of FIG. 1 at the propelling system thereof.

FIG. 4B is another side view diagram of the capsule of FIG. 1 at the propelling system thereof.

FIG. 5A is a side view diagram of a capsule according to the second exemplary embodiment of the disclosure.

FIG. 5B is another side view diagram of a capsule according to the second exemplary embodiment of the disclosure.

FIG. 6 is a top view diagram of a capsule according to the third exemplary embodiment of the disclosure.

FIG. 7 is a top view diagram of a capsule according to the fourth exemplary embodiment of the disclosure.

FIG. 8 is a side view diagram of a capsule according to the fifth exemplary embodiment of the disclosure.

FIG. 9A is a side view diagram of the capsule of FIG. 8 at the propelling system thereof.

FIG. 9B is another side view diagram of the capsule of FIG. 8 at the propelling system thereof.

FIG. 9C is further another side view diagram of the capsule of FIG. 8 at the propelling system thereof.

FIG. 10 is a side view diagram of a capsule according to the sixth exemplary embodiment of the disclosure.

FIG. 11 is a side view diagram of a capsule according to the seventh exemplary embodiment of the disclosure.

FIG. 12 is a side view diagram of a capsule according to the eighth exemplary embodiment of the disclosure.

FIG. 13 is a back view diagram of the capsule of FIG. 13.

FIG. 14 is a side view diagram of a capsule according to the ninth exemplary embodiment of the disclosure.

### DESCRIPTION OF THE EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to improve the understanding of the drawing.

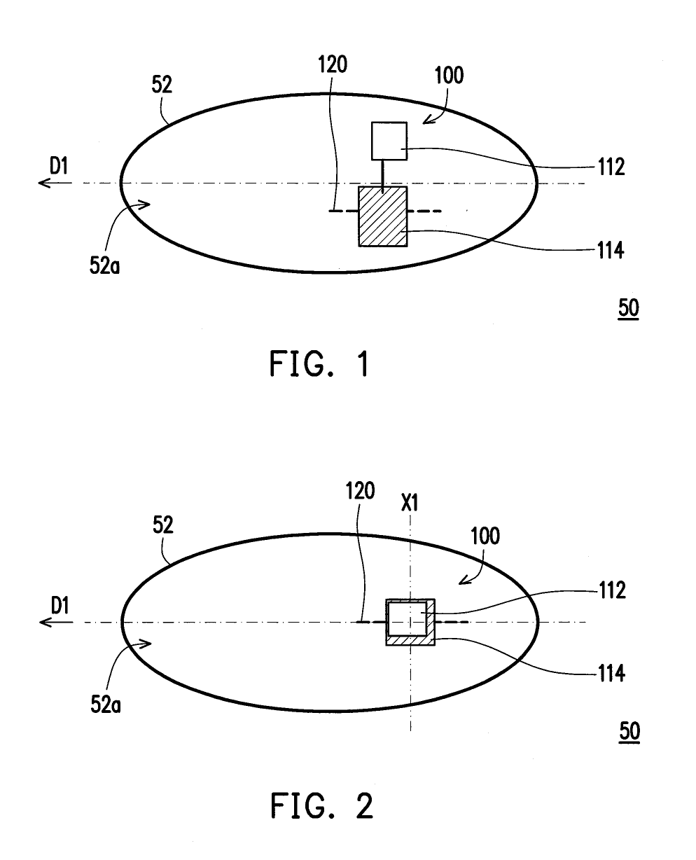

FIG. 1 is a side view diagram of a capsule according to the first exemplary embodiment of the disclosure and FIG. 2 is the top view diagram of the capsule of FIG. 1. Referring to FIGS. 1 and 2, in the embodiment, a capsule 50 includes a shell 52 and a propelling system 100. The shell 52 has a chamber 52a and the propelling system 100 is disposed in the chamber 52a. The propelling system 100 includes a mass 112 and a damping module 120. The mass 112 is configured for vibrating in the chamber 52a along a plurality of directions. The damping module 120 is coupled between the mass 112 and the capsule 50 for absorbing, in one or more directions, the kinematic energy of the mass 112. The damping module 120 provides the smallest damping effect on a first direction D1 among the multiple directions, so that the propelling system 100 is able to drive the capsule 50 moving forward towards the first direction D1.

In more details, in the embodiment, the mass 112 is driven by a dynamic device 114 in the chamber 52a, to vibrate along multiple directions. In the embodiment, the dynamic device 114 includes a motor having a rotation shaft X1. The mass 112 is eccentrically disposed on the rotation shaft X1, as shown in FIG. 2. In other embodiments of the present application, the mass can also be made of an inhomogeneous material and having an asymmetric mass distribution.

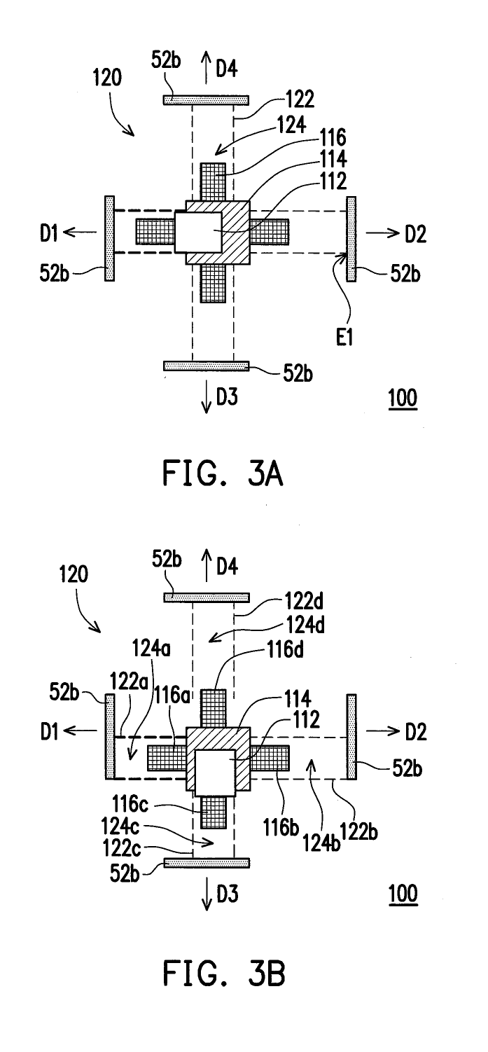

FIGS. 3A and 3B are top view diagrams of the capsule of FIG. 1 at the propelling system thereof. In the embodiment, the damping module 120 includes a plurality of damping elements 122 disposed in different directions and coupled between the mass 112 and the capsule 50. Each of the damping element 122 is provided with a channel 124 along the corresponding vibration direction, and the mass 112 is provided with a plurality of guiding rods 116 attached to the dynamic device 114, wherein each guiding rod 116 is located in the corresponding channel 124, so that when the mass 112 vibrates, the damping elements 122 can absorb the vibrations of the mass 112. In the embodiment, the damping element part of the damping module 120 can be a helical spring, but in other embodiments of the disclosure, the damping element 122 can be other elastic parts such as elements made of elastomer or rubber.

When the mass 112 vibrates in the chamber 52a along the possible directions, the damping elements 122 respectively disposed on the directions are accordingly compressed or stretched with the vibration of the mass 112 to store and absorb the kinematic energy of the mass 112 for providing buffering. In addition, the first end El of each of the damping elements 122 is coupled to the capsule 50 and left detached from the mass 112. The mass 112 remains in location thanks to the guiding rods 116 attached to the dynamic device 114. The damping elements 122 are allowed to slide alongside the sliding rails 52b attached to the inner walls of the chamber 52a in a direction perpendicular to the longitudinal axis of each damping elements 122. When some of the damping elements 122 are compressed or stretched during the vibration of the mass 112, the other damping elements 122 are prevented to be compressed or stretched in the directions departing from the vibration direction, and the skew caused by the eccentric compression or stretching can be accordingly prevented as well.

In more details, in the embodiment, the damping module 120 includes four damping elements 122a-122d respectively disposed on four directions D1-D4, as shown in FIG. 3B. FIG. 3B shows the mass 112 having shifted toward the bottom due to the inertia of the mass 112, the damping elements 122d being elongated while the damping element 122c being compressed, the mass 112 being allowed to slide in such a direction thanks to the sliding rails 52b. The damping elements 122a-122d are respectively coupled to the mass 112 and the capsule 50 and include four channels 124a-124d corresponding to the vibration directions D1-D4; the four guiding rods 116a-116d attached to the mass 112 are respectively located in the corresponding channels 124a-124d. In addition, the damping elements 122a-122d are allowed to slide alongside the sliding rails 52b of the chamber 52a in a direction perpendicular to the longitudinal axis of the damping elements 122 allowed to slide along the inner-wall of the chamber 52a, so that the damping elements 122a-122d can move along the inner-wall of the chamber 52a. Thus, FIGS. 3A and 3B illustrate the situations corresponding to prior and after the movement of the mass 112 towards the third direction D3.

Referring to FIG. 3A first and then FIG. 3B, the mass 112 moves towards the third direction D3, which makes the damping elements 122a-122d originally in a rest state change their states: the damping element 122c is compressed, the damping element 122d is stretched and the damping elements 122a and 122b can move along the sliding rails 52b. As a result, the damping elements 122a and 122b are subject to less lateral forces when the mass 112 moves along directions D3 and D4.

It should be noted that, the damping ratio of the damping element 122a located on the first direction D1 is lower than the damping ratio of the rest of the damping elements 122b-122d so that the damping module 120 can provide the smallest damping effect on the first direction D1. Because the damping element 122a damping action is minimal or even eliminated, by action of reaction, the whole capsule 50 moves forward towards the first direction D1.

When the mass tends to move toward the opposite direction of D1, namely D2, the mass is free to move away from the damping element 122a, thus the previous motion along D1 is not eliminated as if damping element 122a was attached to the mass 110.

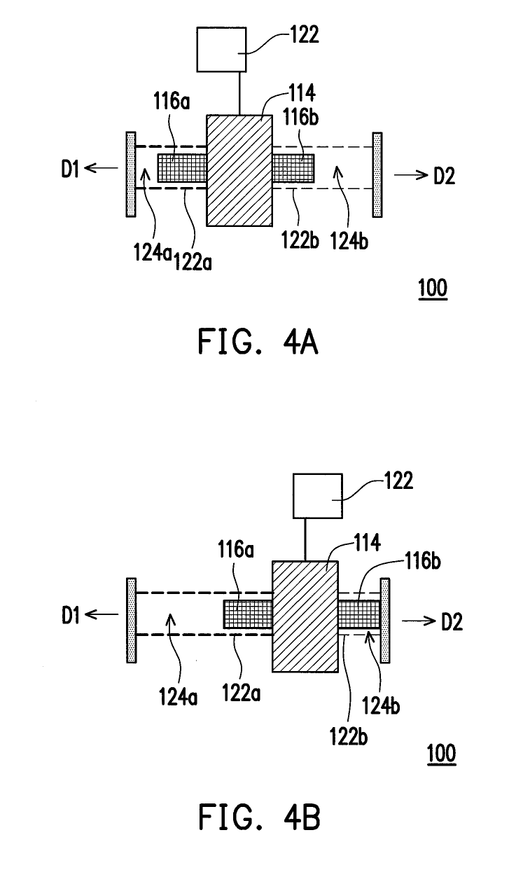

In more details, FIGS. 4A and 4B are provided for understanding. FIGS. 4A and 4B are side view diagrams of the capsule of FIG. 1 at the propelling system thereof Referring to FIGS. 4A and 4B, only the first direction D1 and the second direction D2 among the four directions of FIGS. 3A and 3B are shown, and the third direction D3 and the fourth direction D4 in FIGS. 3A and 3B can be considered as the two directions out of the plane of the page and towards the plane of the page in FIGS. 4A and 4B.

In the embodiment, the damping ratio of the damping element 122a located on the first direction D1 is smaller than the damping ratio of the rest of the damping elements 122b-122d. When the mass 112 moves towards the first direction D1, as shown by FIG. 4A, since the damping ratio of the damping element 122a is smaller than the damping ratio of the damping element 122b, the deformation amount of the damping element 122a is less than the deformation amount of the damping element 122b, and therefore, the propelling system 100 is driven to have a larger displacement towards the first direction D1 than the second direction, because the kinetic energy of the mass 112 is not dampened. The damping element 122a can also be replaced by a rigid element of cylindrical shape. On the contrary, when the mass 112 moves towards the second direction D2, as shown by FIG. 4B, since the deformation amount of the damping element 122b is greater than the deformation amount of the damping element 122a, and therefore, the propelling system 100 is driven to have a smaller displacement towards the second direction D2.

Based on the above-mentioned principle, when the mass 112 vibrates in multiple directions, with the damping element 122a having the smallest damping ratio being disposed on the first direction D1, the propelling system 100 produces the largest displacement towards the first direction D1 so that the resulting displacement of the capsule 50 is a proceeding motion towards the first direction D1.

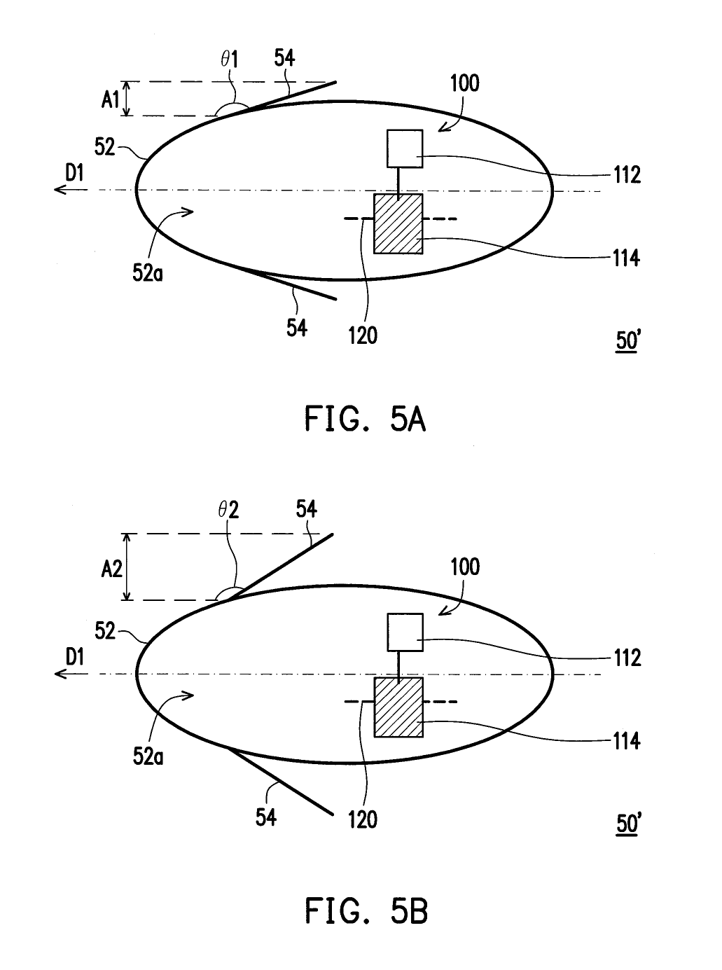

FIGS. 5A and 5B are side view diagrams of a capsule according to the second exemplary embodiment of the disclosure. Referring to FIGS. 5A and 5B, in the embodiment, in addition to the parts of the above-mentioned capsule 50, the capsule 50′ further includes a plurality of flaps 54 disposed on the external surface of the capsule 50′, and the included angle between each of the flaps 54 and the first direction D1 is greater than 90° but lower than 180°.

In more details, referring to FIG. 5A, in the embodiment, when the propelling system 100 propels the capsule 50′ moving forward towards the first direction D1, the flap 54 and the first direction D1 maintain an included angle θ1 therebetween so that the capsule 50′ can continuously move forward toward the first direction D1 during the propelling of the propelling system 100. Herein, the propelling system 100 serves as a navigation apparatus.

Referring to FIG. 5B, when the propelling system 100 serving as the navigation apparatus tends to move in a direction opposite to D1, the flap 54 and the first direction D1 maintain another included angle θ2 therebetween, which is the maximum angle the flap 54 allowed to make with the direction D1.

As a result, when the included angle between the flap 54 and the first direction D1 is θ2, the forward movement of the capsule 50′ towards the first direction D1 would be hindered. For example, the speed of the forward movement of the capsule 50′ towards the opposite of the direction D1 becomes slower or the capsule 50′ stops moving backward, that is opposite to the first direction D1. In other words, by disposing the flaps 54, it is helpful to keep the capsule 50′ moving forward towards the first direction D1. The material of the flaps 54 is non rigid so as not to potentially harm the body of the patient. The flaps 54 can be used with or without the mass 112, if a fluid is allowed to move in both directions, such as for example in peristalsis, the flaps 54 will favour a motion in one direction more than the opposite direction.

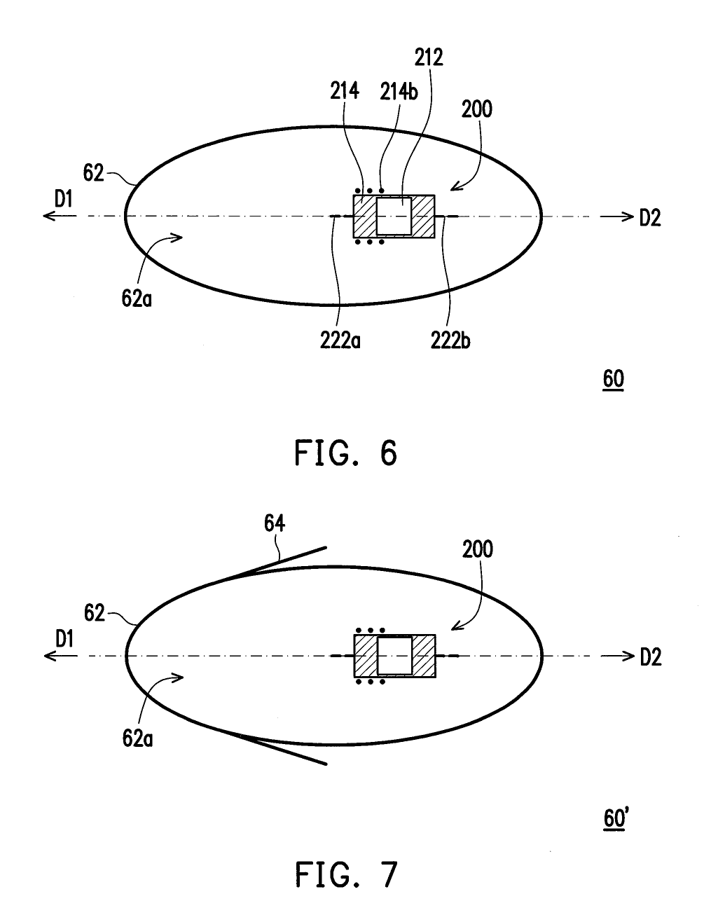

FIG. 6 is a top view diagram of a capsule according to the third exemplary embodiment of the disclosure. Referring to FIG. 6, in the embodiment, the capsule 60 includes a shell 62 and a propelling system 200. The shell 62 has a chamber 62a and the propelling system 200 is disposed in the chamber 62a. The propelling system 200 includes a mass 212 and a damping module 220. The mass 212 is configured for vibrating in the chamber 62a along a plurality of directions. In the embodiment, the mass 212 makes reciprocating motion along the first direction D1 and a second direction D2 opposite to the first direction D1.

In more details, the mass 212 is driven by a dynamic device 214. In the embodiment, the dynamic device 214 has a coil 214b for driving the mass 212, which can be a magnet with poles positioned along the axis made of both directions D1 and D2. The mass 212 can also be a material which can be attracted by a magnetic force but which doesn't significantly remain magnetized after the magnetic force is removed. Some ferromagnetic materials respond to this property, such as iron. The dynamic device 214 can drive the mass 212 to move in the chamber 62a along the first direction D1 and the second direction D2.

Further referring to FIG. 6, in the embodiment, the damping module 220 is coupled between the mass 212 and the capsule 60 for absorbing the kinematic energy of the mass 212, said absorption being privileged in one direction versus the other. Namely, the damping module 220 absorbs kinetic energy along direction D2 and absorbs little or no energy in direction D1, allowing the mass 212 to propel the capsule 60 in direction D1. The damping module 220 includes a plurality of damping elements 222a and 222b respectively disposed on the first direction D1 and the second direction D2 and coupled between the mass 110 and the capsule 60. The damping elements 222a and 222b are compressed or stretched with the vibration of the mass 212 to store and absorbs the kinematic energy of the mass 212, depending on the direction of vibration of the mass 212.

The damping ratio of the damping element 222a located on the first direction D1 is smaller than the damping ratio of the damping element 222b so that the damping module 220 can provide the smallest damping effect on the first direction D1. Due to the result, when the mass 212 moves along the first direction D1 and the second direction D2, the deformation amount of the damping element 222a with a smaller damping ratio is less than the deformation amount of the damping element 222b. The damping elements 222a and 222b are detached from the mass 212, in such a manner that when the mass 212 moves in the direction D2 while damping element 222b, it doesn't pull along damping element 222a. Similarly, when the mass 212 moves in direction D1, thus entering in contact with damping element 222a, it doesn't pull along the element 222b.

Accordingly, when the mass 212 moves towards the first direction D1, the propelling system 200 has a larger displacement towards the first direction D1. On the contrary, when the mass 212 moves towards the second direction D2, the propelling system 200 produces a smaller displacement towards the second direction D2. Based on the above-mentioned principle, by disposing the damping element 222a with the smallest damping ratio on the first direction D1, the propelling system 200 can produce the largest displacement towards the first direction D1 and moreover, the resultant displacement of the capsule 60 is moving forward towards the first direction D1.

FIG. 7 is a top view diagram of a capsule according to the fourth exemplary embodiment of the disclosure. Referring to FIG. 7, on the other hand in the embodiment, in addition to the parts of the above-mentioned capsule 60, the capsule 60′ further includes a plurality of flaps 64 disposed on the external surface of the capsule 60′, and the included angle between each of the flaps 64 and the first direction D1 is greater than 90° but less than 180°.

The function of the flaps 64 in the capsule 60′ is the same as the function of the flaps 54 in the capsule 50′ of the second embodiment. Therefore, when the propelling system 200 propels the capsule 60′ moving forward towards the first direction D1, by keeping the included angle between the flap 64 and the first direction D1 within the above-mentioned range of angle, it can assist the capsule 60′ in moving forward towards the first direction D1 or hinder the capsule 60′ from moving backward, that is towards the second direction D2. In other words, disposing the flaps 64 can be helpful for the capsule 60′ to move forward towards the first direction D1.

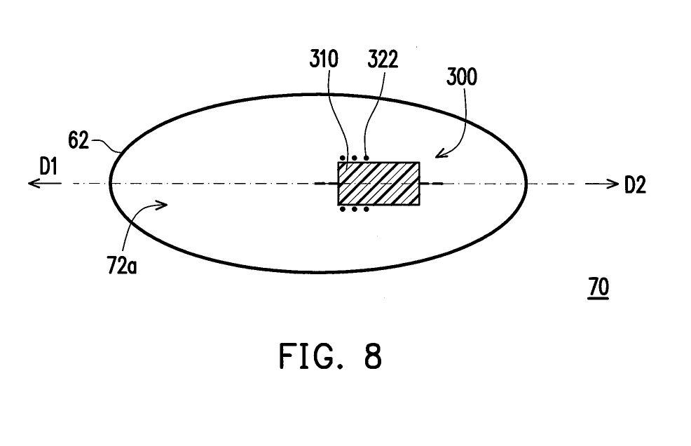

FIG. 8 is a side view diagram of a capsule according to the fifth exemplary embodiment of the disclosure. Referring to FIG. 8, the capsule 70 in the embodiment includes a shell 72 and a propelling system 300. The shell 72 has a chamber 72a and the propelling system 300 is disposed in the chamber 72a. The propelling system 300 includes a mass 310 and a damping module 320. The mass 310 is made of magnetic material. In the embodiment, the mass 310 can be a magnetic-iron block, but in other embodiments, the mass 310 can be made of other magnetic materials, such as rare earth magnets or material which can be attracted by a magnetic force but which doesn't significantly remain magnetized after the magnetic force is removed. Some ferromagnetic materials respond to this property, such as iron, which the disclosure is not limited to. The damping module 320 includes a magnetic power source 322 for driving the mass 310 to make reciprocating motion along a first direction D1 and a second direction D2 relatively to the first direction D1.

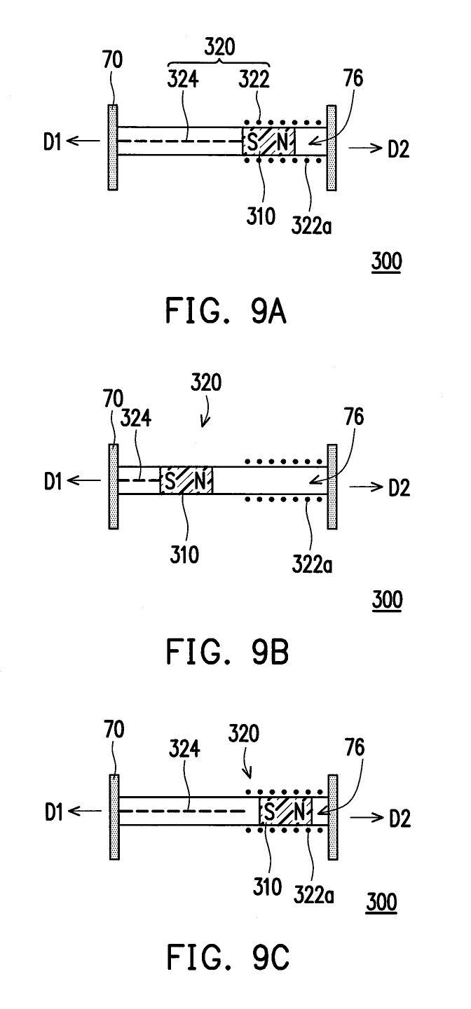

FIG. 9A is a side view diagram of the propelling system of the capsule of FIG. 8. Referring to FIG. 9A, in the embodiment, the electro-magnetic power source 322 includes an electromagnetic coil 322a disposed in the second direction D2, and the damping module 320 further includes a damping element 324 disposed in the first direction D1 and connected between the mass 310 and the capsule 70. In addition, the capsule 70 possesses a channel 76 and the electro-magnetic power source 322 is allowed to drive the mass 310 to make reciprocating motion along the first direction D1 and the second direction D2 in the channel 76. In addition, the magnetic power source 322 associated with the damping element 324 can store and absorb the kinematic energy of the mass 310. The damping element 324 makes contact to the mass 310 but is not attached to it.

FIGS. 9B and 9C are side view diagrams of the propelling system of FIG. 8. Referring to FIGS. 9A-9C, in more details, the electromagnetic coil 322a is disposed on the second direction D2 of the channel 76, while the damping element 324 is disposed on the first direction D1 of the channel 76 and connected between the mass 310 and the capsule 70. When the mass 310 is in balance state, the electromagnetic coil 322a can drive the mass 310 to move towards the first direction D1 along the channel 76, as shown by FIG. 9A.

Then, when the mass 310 moves along the channel 76 towards the first direction D1, the damping element 324, pushed by the mass 310, changes its state from balance state to compression state, as shown by FIG. 9B. Finally, when the electromagnetic coil 322a is no more powered so as to release the compressed damping element 324, the resuming force of the damping element 324 pushes the mass 310 to fast move along the channel 76 towards the second direction D2 and then, the damping element 324 returns its balance state again. At the time, the magnetic power source 322 can provide a damping action, as shown by FIG. 9C. When the above-mentioned actions are repeated, the magnetic power source 322 is able to drive the mass 310 making reciprocating motion along the first direction D1 and the second direction D2, and privileging a motion along direction D1 over direction D2.

In addition, by using an external signal to control the electromagnetic coil 322a, the damping effect provided by the electromagnetic coil 322a can be greater than the damping effect provided by the damping element 324, which thereby makes the damping module have the smallest damping effect on the first direction D1. Based on the above-mentioned principle, the propelling system 300 can make the driven capsule 70 produce a resulting displacement forward towards the first direction D1, and the proceeding speed of the capsule 70 can be changed according to the acceleration variation of the propelling system 300.

On the contrary, if the external signal controls the electromagnetic coil 322a to produce a damping effect less than the damping effect provided by the damping element 324, the damping module has the smallest damping effect on the second direction D2. Therefore, the propelling system 300 can make the driven capsule 70 produce a resulting displacement forward towards the second direction D2.

It should be noted that the damping module 320 of the capsule 70 in the embodiment is not limited by the above-mentioned structures, and two more embodiments similar to the capsule 70 are described in following.

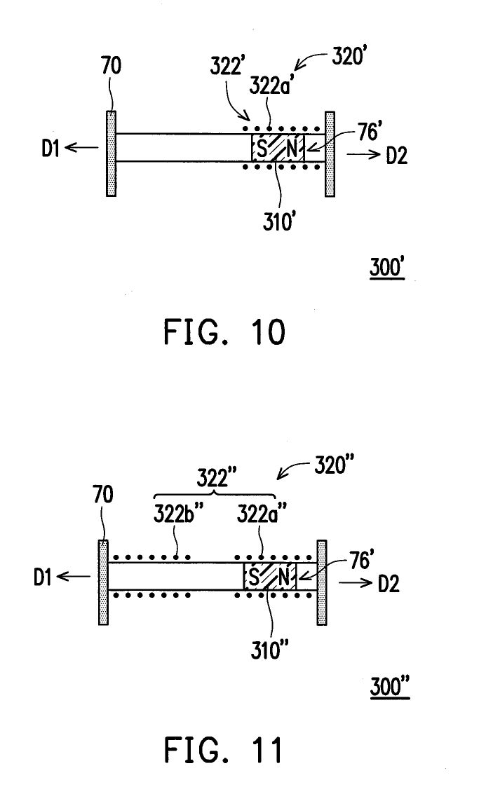

FIG. 10 is a side view diagram of a capsule according to the sixth exemplary embodiment of the disclosure. Referring to FIG. 10, the damping module 320′ of a propelling system 300′ in the embodiment does not include the damping element 324, in which an electromagnetic coil 322a′, part of a magnetic power source 322′, disposed on the second direction D2 of a channel 76′ drives a mass 310′ to make reciprocating motion along the first direction D1 and the second direction D2 in the channel 76′. The electromagnetic coil 322a can be located at any position along the channel 76′. The magnetic power source 322′, part of the damping module 320′, can also absorb the kinematic energy of the mass 310′ during the vibration of the mass 310′.

If an external signal controls the electromagnetic coil 322a′ to provide the smallest damping effect on the first direction D1, the resulting displacement of the capsule 70′ driven by the propelling system 300′ is forward toward the first direction D1. On the contrary, if the external signal controls the electromagnetic coil 322a′ to provide the smallest damping effect on the second direction D2, the resulting displacement of the capsule 70′ driven by the propelling system 300′ is forward towards the second direction D2.

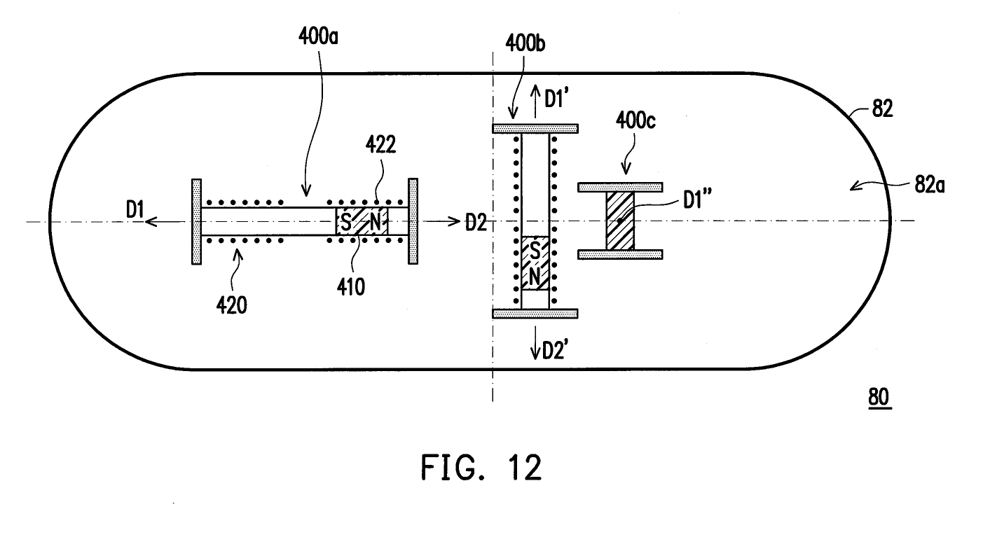

FIG. 11 is a side view diagram of a capsule according to the seventh exemplary embodiment of the disclosure. Referring to FIG. 12, in the embodiment, the damping module 320″ of a propelling system 300″ does not include the damping element 324; however, the magnetic power source 322″ comports a first and a second electromagnetic coil 322a″ and 322b″ disposed on the second direction D2 of a channel 76″, disposed along the directions D1 and D2. The magnetic power source 322″, part of the damping module 320″, can drive a mass 310″ to make reciprocating motion along the first direction D1 and the second direction D2 in the channel 76″, and can also absorb the kinematic energy of the mass 310″ during the vibration of the mass 310″.

If an external signal controls the first electromagnetic coil 322a″ and the second electromagnetic coil 322b″ to provide the smallest damping effect on the first direction D1, the resulting displacement of the capsule 70″ driven by the propelling system 300″ is forward towards the first direction D1. On the contrary, if the external signal controls the first electromagnetic coil 322a″ and the second electromagnetic coil 322b″ to provide the smallest damping effect on the second direction D2, the resulting displacement of the capsule 70″ driven by the propelling system 300″ is forward towards the second direction D2.

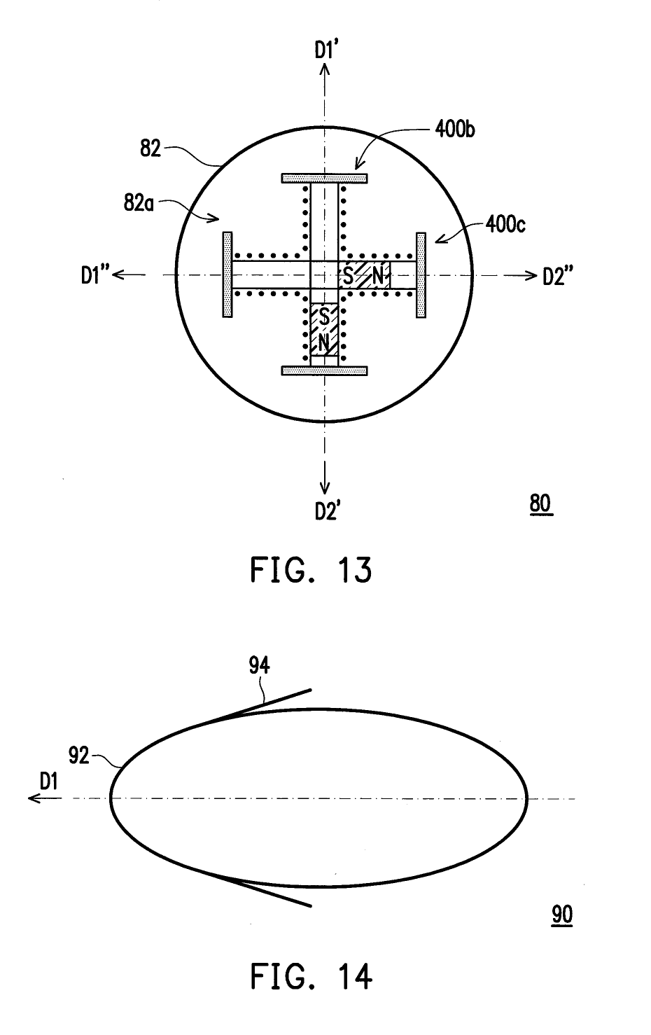

FIG. 12 is a side view diagram of a capsule according to the eighth exemplary embodiment of the disclosure and FIG. 13 is a back view diagram of the capsule of FIG. 12. Referring to FIGS. 12 and 13, in the embodiment a capsule 80 includes a shell 82 and a plurality of propelling systems 400a, 400b, 400c, in which the shell 82 has a chamber 82a and the propelling systems 400a, 400b, 400c are disposed in the chamber 82a. The layout of the propelling systems 400a, 400b, 400c makes the first direction D1, D1′, D1″ of each of the propelling systems 400a, 400b, 400c different from the first directions D1, D1′, D1″ of the rest of the propelling systems 400a, 400b, 400c.

In more details, each of the propelling systems 400a, 400b, and 400c includes a mass 410 and a damping module 420. The mass 410 is configured for vibrating in the chamber 82a along a plurality of directions. The damping module 420 is coupled between the mass 410 and the capsule 80 and includes a magnetic power source 422 for absorbing the kinematic energy of the mass 410. The magnetic power source 422 drives the mass 410 to make reciprocating motion along the first direction D1 and a second direction D2, in which the damping module 420 provides the smallest damping effect on the first direction D1 among a plurality of directions.

It can be seen from the above-mentioned fifth, sixth and seventh embodiments that by appropriately controlling a control signal sent to the magnetic power sources 322, 322′ and 322″, each propelling system 300, 300′ and 300″ can be controlled to provide a motion along D1, D1′ or D1″ or D2, D2′ or D2″ or any combination of these directions.

In this embodiment, the capsule 80 includes three propelling systems 400a-400c, which the disclosure is not limited to. The shell 82 of the capsule 80 has a chamber 82a and the three propelling systems 400a-400c are disposed in the chamber 82a. The propelling systems 400a-400c respectively have first directions D1, D1′ and D1″ and second directions D2, D2′ and D2″. The layout of the propelling systems 400a-400c makes the first directions D1, D1′ and D1″ of the propelling systems 400a-400c, i.e., the resultant displacement directions of the propelling systems 400a-400c, different from each other, so that the capsule 80 can forward move towards multiple directions through the action of the propelling systems 400a-400c. In this embodiment, the capsule 80 can therefore move along 3 different directions each perpendicular to each other, such as the 3 direction x, y, z of a referential system.

FIG. 14 is a side view diagram of a capsule according to the ninth exemplary embodiment of the disclosure. Referring to FIG. 15, in the embodiment, the capsule 90 includes a shell 92, but does not include the propelling system. By using the existing environment of the capsule 90 to provide an external propelling force, the capsule 90 can be propelled to move forward towards the first direction D1. In addition, the capsule 90 includes a plurality of flaps 94 disposed on the external surface of the capsule 90, in which the included angle between each of the flaps 94 and the first direction D1 is greater than 90° but less than 180°.

The function of the flaps 94 in the capsule 90 is the same as the function of the flaps 54 in the capsule 50′ of the second embodiment or the function of the flaps 64 in the capsule 60′ of the fourth embodiment. Therefore, when the capsule 90, driven by the external propelling force, moves forward towards the first direction D1, by keeping the included angle between the flap 94 and the first direction D1 within the above-mentioned range, it can assist the capsule 90 in moving forward towards the first direction D1 or hinder the capsule 90 from moving backward towards the second direction D2. In other words, disposing the flaps 94 is helpful for the capsule 90 to move forward towards the first direction D1.

In summary, the disclosure provides a propelling system, suitable to be disposed in a chamber of a capsule. The propelling system has a mass disposed in the chamber along at least one direction, and a damping module is employed for absorbing the kinetic energy of the mass in one direction while it doesn't absorb the kinetic in another direction, hence allowing the apparatus to be propelled in the direction where the kinetic energy of the mass is not dampened. In this way, the propelling system can produce the propelling effect without using an external device. By using such propelling system in different direction, the capsule can therefore be steered in various directions.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims.

What is claimed is:

1. A propelling system, disposed in a chamber of a capsule and comprising:
a mass, configured for vibrating in the chamber along a plurality of directions; and
a damping module, coupled between the mass and the capsule for absorbing kinetic energy of the mass, wherein the damping module provides the smallest damping effect along a first direction, one of the plurality of directions.

2. The propelling system as in claim 1, wherein the damping module comprises a plurality of damping elements respectively disposed on the plurality of directions and coupled between the mass and the capsule, wherein a damping ratio of a first damping element, one of the plurality of damping elements, along the first direction is smaller than a damping ratio of the remaining of the plurality of damping elements.

3. The propelling system as in claim 2, wherein each of the plurality of damping elements comprises a channel along the corresponding vibration direction, and the mass is provided with a plurality of guiding rods respectively located in the corresponding channels.

4. The propelling system as in claim 2, wherein a first end of each of the plurality of damping elements is coupled to the capsule and allowed to slide along the inner walls of the chamber of the capsule.

5. The propelling system as claimed in claim 2, wherein each of the plurality of damping elements comprises a spring, or an elastomer material, or an elastic material.

6. The propelling system as claimed in claim 1, further comprising:
a dynamic device, configured for driving the mass to vibrate in the chamber along the plurality of directions.

7. The propelling system as in claim 6, wherein the dynamic device comprises a motor having a rotation shaft, and the mass is eccentrically disposed on the rotation shaft.

8. The propelling system as in claim 1, wherein the mass has an asymmetric weight distribution.

9. The propelling system as in claim 1, wherein the mass makes reciprocating motion along two opposite directions.

10. The propelling system as claimed in claim 9, further comprising a magnetic power source for driving the mass to make reciprocating motion along the first direction and a second direction opposite to the first direction, wherein the mass is made of a magnetic material and wherein the magnetic power source and the mass act as the damping module.

11. The propelling system as claimed in claim 10, wherein the magnetic power source comprises an electromagnetic coil disposed around a channel.

12. The propelling system as claimed in claim 11, wherein the damping module further comprises a damping element disposed along the first direction and coupled between the mass and the capsule.

13. The propelling system as in claim 10, wherein the magnetic power source comprises a first electromagnetic coil and a second electromagnetic coil disposed along one direction.

14. The propelling system as in claim 1, further comprising a plurality of flaps disposed on an external surface of the capsule, wherein each of the flaps has an included angle towards the direction greater than 90° but less than 180°.

15. A capsule, comprising:
a shell, having a chamber; and
at least one propelling system, disposed in the chamber, wherein the at least one propelling system comprises:
a mass, vibrating in the chamber along a plurality of directions; and
a damping module, coupled between the mass and the capsule for absorbing kinetic energy of the mass, wherein the damping module provides the smallest damping effect along a first direction.

16. The capsule as in claim 15, wherein the damping module comprises a plurality of damping elements respectively disposed on the directions and coupled between the mass and the capsule, wherein a damping ratio of a first damping element along the first direction is smaller than a damping ratio of the remaining damping elements.

17. The capsule as claimed in claim 16, wherein each of the plurality of damping elements comprises a channel along the corresponding vibration direction and the mass is provided with a plurality of guiding rods respectively located in the corresponding channels.

18. The capsule as claimed in claim 16, wherein a first end of each of the plurality of damping elements is coupled to the capsule and allowed to slide along the inner walls of the chamber of the capsule.

19. The capsule as claimed in claim 16, wherein each of the plurality of damping elements comprises a helical spring, a metallic spring, an elastomer material, or an elastic material.

20. The capsule as claimed in claim 15, further comprising:
a dynamic device, configured for driving the mass to vibrate in the chamber along the plurality of directions.

21. The capsule as claimed in claim 20, wherein the dynamic device comprises a motor having a rotation shaft, and the mass is eccentrically disposed on the rotation shaft.

22. The capsule as claimed in claim 15, wherein the mass makes reciprocating motion along two opposite directions.

23. The capsule as claimed in claim 22, comprising a magnetic power source for driving the mass to make reciprocating motion along the first direction and a second direction opposite to the first direction, wherein the mass is made of a magnetic material and wherein the magnetic power source and the mass act as the damping module.

24. The capsule as claimed in claim 23, wherein the magnetic power source comprises an electromagnetic coil disposed around a channel.

25. The capsule as claimed in claim 24, wherein the damping module further comprises a damping element disposed along the first direction and coupled between the mass and the capsule.

26. The capsule as claimed in claim 23, wherein the magnetic power source further comprises a first electromagnetic coil and a second electromagnetic coil disposed along one direction.

27. The capsule as claimed in claim 15, further comprising a plurality of flaps disposed on an external surface of the capsule, wherein each of the flaps has an included angle towards the first direction greater than 90° but less than 180°.

28. The capsule as in claim 15, comprising at least two propelling systems, each propelling system having a privileged propagation direction different from the others.
